(19) United States
(12) Patent Application Publication
(10) Pub. No.: US 2006/0165555 A1
(43) Pub. Date: Jul. 27, 2006

(54) System, method, and apparatus for an intense ultraviolet radiation source

(75) Inventors: Rick B. Spielman, Tijeras, NM (US); Jean-Francois Leon, Le Bourg (FR); Michael L. Fraim, Corralas, NM (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: ABQ Ultraviolet Pollution Solutions, Inc., Albuquerque, NM (US)

(21) Appl. No.: 11/372,523

(22) Filed: Mar. 10, 2006

Related U.S. Application Data
(63) Continuation of application No. 10/218,998, filed on Aug. 15, 2002, now abandoned.
(60) Provisional application No. 60/312,088, filed on Aug. 15, 2001.

(51) Int. Cl. A61L 9/20

(52) U.S. Cl. 422/4; 422/24

(57) ABSTRACT

An intense ultraviolet radiation source is disclosed that may be operated in substantially any arbitrary gas environment, without regard to a containment envelope for the ultraviolet radiation source. The intense ultraviolet radiation source can be generated by applying a pulsed or continuous electrical discharge to a partially ionized combustion flame via two electrodes. The combustion flame and electrical discharge can be focused, contained, or confined by gas pressure, electric fields, and/or magnetic fields. Optionally, the thermal energy in the flame and the electrical discharge power input may be augmented with an electromagnetic radiation source, such as a radio-frequency induction heater, a laser, or a microwave generator. Impurities may be placed in contact with or added to the fuel and/or the oxidizer to further alter the emitted ultraviolet radiation spectral brightness as needed. The ultraviolet source may be applied to the molecular dissociation of pollutants in exhaust gas streams of combustion systems. The efficient dissociation of such pollutants requires UV in the wavelength ranges of the vacuum ultraviolet band (100 nm-180 nm) and the UV-C band (180 nm-280 nm).

### CROSS-REFERENCE TO RELATED APPLICATION

This application claims priority under 35 U.S.C. § 1.119(e) from U.S. patent application Ser. No. 60/312,088, filed Aug. 15, 2001, which is incorporated herein by reference, to the extent it is consistent with this invention and this application.

### FIELD OF THE INVENTION

This invention relates generally to the field of ultraviolet radiation sources, and more particularly to an efficient, low cost ultraviolet radiation source that can operate under atmospheric conditions while exposed to the environment. Such operation provides for greater intensity of the ultraviolet radiation emanating from the source, allowing for more effective use of the ultraviolet radiation in many different applications as, for example, molecular dissociation of pollutants in exhaust gas streams of combustion systems. This invention also relates to the field of molecular dissociation and the chemical reaction of pollutants in gaseous waste streams. A system and method for an ultraviolet radiation source is disclosed, as well as a system and method for molecular dissociation and the chemical reaction of pollutants in exhaust gas streams of combustion systems.

### BACKGROUND OF THE INVENTION

Ultraviolet radiation (UV) is the electromagnetic radiation having wavelengths between that of x rays and visible light, with wavelengths of about 100 nanometers (nm) to about 400 nm. Ultraviolet radiation can be further divided into spectral bands: UV-A, having wavelengths in the range of about 320 nm to about 400 nm, UV-B, having wavelengths of about 290 nm to about 320 nm, and UV-C, having wavelengths between 290 nm and 180 nm. The shorter the wavelength of the ultraviolet radiation, the higher the photon energy, and the more useful the radiation is for molecular dissociation. A number of important uses for ultraviolet radiation have been found. These uses have driven the development of ultraviolet radiation sources that produce the desired wavelengths for the particular applications. For example, ultraviolet light with wavelengths of about 245 nm has been used as a germicidal aid in purifying wastewater as these wavelengths of UV can kill or alter the genetic material of certain organisms rendering them unable to reproduce, and thus suppressing microorganism growth. Other applications of ultraviolet radiation include the dissociation of pollutants such as VOCs (Volatile Organic Compounds) and TAPs (Toxic Air Pollutants) with UV at about 185 nm. Examples of VOCs and TAPs include, among others, trichloroethylene, benzene, and acrylonitrile. Many of these applications involve the application of ultraviolet radiation to contaminated streams in contact with some photocatalyst, causing the molecular dissociation of those contaminants.

Commercial ultraviolet radiation sources require closed or sealed systems to produce the desired spectrum of wavelength because they may utilize a unique mix of rare gases, such as mercury vapor, xenon, deuterium, or krypton, or may have to operate at some pressure other than atmospheric. A UV-transmissive “envelope” must be used to contain these systems, which may be made of, for example, quartz or sapphire. One drawback to the envelopes is that they absorb some of the UV radiation emanating from the source, particularly the shorter higher energy wavelengths, thus reducing the amount of useful UV radiation that is emitted and reducing the overall efficiency of the system. As these envelopes (typically sapphire or quartz) absorb energy they become heated, which in turn causes an increase in the absorption coefficient of the envelope. If the external cooling cannot keep up with the heating, the envelope will fail. With gas cooling, most UV-C bulb's outputs are limited to 10 W per meter length. Although the filtering provided by the envelopes may be necessary in such applications as mercury vapor lamps, where human exposure to UV radiation is undesirable, the envelopes also absorb the desired lower energy, longer wavelength UV radiation as well. Another drawback to a UV-transmissive envelope is that the envelope is slowly coated with electrode material thereby gradually reducing the overall transparency of the envelope in.time to all wavelengths of light.

Another drawback to current UV generating systems is that the UV sources or bulbs must be replaced as a unit. As the-electrodes wear, the envelope is coated, the gases become contaminated, and the efficiency of the source decreases until replacement is required. The cost of these bulbs can be significant for high power applications.

### DISCUSSION OF PRIOR ART

Prior art in the field of ultraviolet radiation sources falls into several groupings, including low-pressure mercury vapor discharge, low-pressure rare gas discharge, high-pressure discharge in sodium (mercury and rare gases), electrode-less lamps driven by radio frequencies and microwaves, thermal emitters, and pulsed plasma sources. Most of these disclosures relate to ultraviolet radiation sources that emit UV continuously in time.

Low-pressure mercury discharge is the standard source of low-power UV-A and UV-B ultraviolet radiation. Several disclosures relate to the development of low-pressure sources, their optimization, and methods for applying these sources to numerous applications. For example, U.S. Pat. No. 4,237,401 issued to Couwenberg and entitled “Low-Pressure Mercury Vapor Discharge Lamp,” describes the optimization of low-pressure mercury vapor lamps for UV generation. U.S. Pat. No. 4,349,765 issued to Brandli and entitled “Ultraviolet Generating Device Comprising Discharge Tube Joined to Two Tubular Envelopes” discloses UV generation in which the output is optimized by the addition of rare gases to the mercury vapor. U.S. Pat. No. 6,387,115 issued to Smolka et al. and entitled “Photodynamic Cylindrical Lamp with Asymmetrically Located Electrodes and Its Use” discloses a low-pressure, high-power UV source with a UV transmissive. glass envelope. The disclosures of each these references are herein incorporated by reference to the extent that they are not inconsistent with this application.

Several disclosures discuss the use of low-pressure rare gas discharges, such as xenon and argon rather than mercury, to generate UV radiation. These gases are selected to improve the source efficiency and to remove the mercury environmental hazard from the UV lamp. For example, U.S. Pat. No. 3,970,856 issued to Mahaffey et al. and entitled “Ultraviolet Light Application” discloses the development of hand-held argon discharge UV lamps. U.S. Pat. No. 4,550,275 issued to O'Loughlin and entitled “High Efficiency Pulse Ultraviolet Light Source” discloses the use of xenon in increasing the efficiency of a low pressure UV source. The disclosures of each these references are herein incorporated by reference to the extent that they are not inconsistent with this application.

Yet other disclosures relate to the use of high-pressure lamps for the generation of visible light for area lighting that can be tuned to generate ultraviolet light. See, for example, U.S. Pat. No. 4,112,335 issued to Gonser et al. and entitled “Rapid Pulse Ultraviolet Light Apparatus” in which high-pressure xenon is used to generate an intense pulsed UV source. U.S. Pat. No. 5,905,341 issued to Ikeuchi et al. and entitled “High Pressure Mercury Ultraviolet Lamp” describes a similar lamp using high-pressure mercury rather than xenon to generate ultraviolet radiation. The high pressure of these lamps requires a relatively thick strong envelope, thereby absorbing a fraction of the short wavelength UV that is generated by the lamp. The disclosures of each these references are herein incorporated by reference to the extent that they are not inconsistent with this application.

Yet other sources disclose the use of low-pressure lamps that do not involve the use of metallic electrodes, and thereby increasing the lifetime of the lamps. Because the erosion of electrodes and the re-deposition of electrode material on the glass envelope are serious lifetime issues, effort has been made to use radio-frequency electric fields or microwaves to drive the lamps. For example, U.S. Pat. No. 4,042,850 issued to Ury et al. and entitled “Microwave Generated Radiation Apparatus” describes the development of a microwave coupled plasma lamp. Also, U.S. Pat. No. 6,265,835 issued to Parra entitled “Energy-Efficient Ultraviolet Source and Method” uses other high frequency sources to generate UV in an electrode-less lamp. The disclosures of each these references are herein incorporated by reference to the extent that they are not inconsistent with this application.

Other sources disclose that any object can emit UV radiation if its temperature is high enough. In U.S. Pat. No. 4,149,086 issued to Nath entitled “UV Irradiation Device”, a tungsten filament is heated to high temperatures as a UV source. The limitation is the rapid vaporization of the filament. However, this concept does not lead to temperatures high enough to generate significant quantities of UV-C radiation. The disclosure of this reference is herein incorporated by reference to the extent that it is not inconsistent with this application.

A number of disclosed UV radiation sources are based on pulsed plasmas. The UV source described-in U.S. Pat. No. 3,978,341 issued to Hoell and entitled “Short Wavelength Ultraviolet Treatment of Polymeric Surfaces” uses a pulsed spark between two electrodes to generate ultraviolet radiation at 83.3 nm to 133.5 nm. The patent describes the method used to energize the spark, which involves a high temperature arc occurring in a low-pressure gas. The energy involved in this discharge is minimal. U.S. Pat. No. 5,945,790 issued to Schaefer and entitled “Surface Discharge Lamp” describes an electrical discharge that takes place over the surface of an insulating material. The material that makes up the discharge comes from the insulating material. The advantage of this configuration is the reproducible electrical discharge that takes place at a lower voltage per unit length of the distance between the electrodes. The presence of the insulating material in intimate contact with the discharge provides a limit on the peak current of the discharge. Finally, U.S. Pat. No. 6,031,241 issued to Silfvast et al. and entitled “Capillary Discharge Extreme Ultraviolet Lamp Source for EUV Microlithography and Other Related Applications” describes the use of capillary electrical discharges to generate high temperature plasmas and intense UV sources. Pulsed current is passed through a very small hole or capillary in an insulating material. The initial electrical discharge can be considered a surface discharge along the sides of the capillary hole. Material from the wall of the capillary is eroded and compresses the discharge to the core of the capillary. The current heats the plasma to high temperatures and creates an efficient UV source. The practical limitation of this concept is that the UV radiation can only be extracted from the ends of the electrical discharge near one of the electrodes. The disclosures of each these references are herein incorporated by reference to the extent that they are not inconsistent with this application.

A large amount of prior art disclosures relate to the use of photocatalysts such as titanium dioxide, silicon dioxide, zirconium dioxide, strontium dioxide, tungsten oxide, molybdenum trioxide, or combinations thereof. These photocatalysts are often contacted with the substance that is to be purified, where the photocatalysts are activated by application of ultraviolet radiation. For example, U.S. Pat. No. 5,779,912 issued to Gonzalez-Martin et al. and entitled “Photocatalytic Oxidation of Organics Using a Porous Titanium Dioxide Membrane and an Efficient Oxidant” discloses a method and apparatus for the photochemical oxidation of organic contaminants in air or water. A photocatalyst, which may be titanium dioxide along with a binary oxide, contacts the fluid containing the organic contaminants, and is activated by exposure to ultraviolet radiation. U.S. Pat. No. 6,080,281 issued to Attia and entitled “Scrubbing of Contaminants from Contaminated Air Streams with Aerogel Materials with Optional Photocatalytic Destruction” discloses activating a metal oxide, such as calcium oxide, magnesium oxide, silicon dioxide, or titanium dioxide that is in contact with a contaminated gas stream by ultraviolet radiation to photo-catalytically destroy the contaminants. The disclosures of each these references are herein incorporated by reference to the extent that they are not inconsistent with this application.

A similar system is disclosed in U.S. Pat. No. 5,045,288 issued to Raupp et al. and entitled “Gas-Solid Photocatalytic Oxidation of Environmental Pollutants” which discusses the destruction of organic contaminants from a gas stream by exposing a solid photocatalyst, such as titanium dioxide, zirconium oxide, antimony oxide, zinc oxide, stannic oxide, cerium oxide, tungsten oxide, and ferric oxide, in contact with the contaminated gas stream to a source of ultraviolet radiation. Other systems disclosing the use of ultraviolet radiation to activate metal oxide photocatalysts, including titanium dioxide, to be used in the destruction of contaminants can be found, for example, in U.S. Pat. No. 4,146,450 issued to Araki et al. and entitled “Methods For Removing Nitrogen Oxides From Nitrogen Oxide-Containing Gases”; U.S. Pat. No. 4,980,040 issued to Lichtin et al. and entitled “Photopromoted Method For Decomposing oxides of Nitrogen into Environmentally Compatible Products”; U.S. Pat. No. 5,480,524 issued to Oeste et al. and entitled “Method and Apparatus For Removing Undesirable Chemical Substances from Gases, Exhaust Gases, Vapors, and Brines”; U.S. Pat. No. 6,179,971 issued to Kittrell et al. and entitled “Two Stage Process and Catalyst for Photocatalytic Conversion of Contaminants”; U.S. Pat. No. 6,221,259 also issued to Kittrell et al. and entitled “Process and Catalyst for Photocatalytic Conversion of Contaminants”; U.S. Pat. No. 5,126,111 issued to Al-Ekabi et al. and entitled “Fluid Purification”; and U.S. Pat. No. 6,241,856 issued to Newman et al. and entitled “Enhanced Oxidation of Air Contaminants on an Ultra-Low Density UV-Accessible Aerogel Photocatalyst.” The disclosures of each of these references are herein incorporated by reference to the extent that they are not inconsistent with this application.

One drawback to each of these systems is that a photocatalyst must be present in the system. Another drawback is that the contaminated stream is segregated from the ultraviolet radiation source, thus reducing the efficiency of the ultraviolet radiation source, as the ultraviolet radiation must pass through the containment wall to activate the photocatalyst, resulting in the absorption of a significant amount of the ultraviolet radiation before it reaches the contaminated stream and photocatalyst. In addition, the lifetime of the catalyst is limited by contamination and replacement costs must be included in routine operational costs.

U.S. Pat. No. 6,168,689 issued to Park et al. and entitled “Method and Apparatus for Cleaning Exhaust Gas Discharged from Internal or External Combustion Engine by Using High Voltage Electric Field,” the disclosure of which is herein incorporated by reference to the extent that it is not inconsistent with this application, discloses direct exposure of NOₓ pollutants (consisting of nitric oxide, NOₓ and nitrogen dioxide, NO₂) contaminants to ozone generated by an ultraviolet radiation source. However, the disclosed system uses ozone created by the exposure of atmospheric oxygen to the ultraviolet radiation to oxidize the NOₓ making the NOₓ soluble in water for easy removal. The UV radiation is generated with a coronal discharge and is limited to longer wavelength UV radiation. The disclosed system does not use ultraviolet radiation to directly cause the molecular dissociation of pollutants such as NO,.

Other prior art exists in the technical area of UV dissociation of gaseous pollutants. These references disclose. numerous, variants of ozone or atomic oxygen generation by UV, production of OH radicals from water by UV, and UV in the presence of ammonia. For example, U.S. Pat. No. 3,984,296 issued to Richards and entitled “System and Process for Controlling Air Pollution” describes the use of ultraviolet radiation to impact the chemistry of exhaust gases. Richards describes the use of UV in the range of 150 nm to 500 nm to modify the chemical structure of pollutants to make them easier to collect or remove in conventional fashions. The disclosure of this reference is herein incorporated by reference to the extent that it is not inconsistent with this application. U.S. Pat. No. 4,097,349 issued to Zenty and entitled “Photochemical Process for Fossil Fuel Combustion Products Recovery and Utilization” discloses many potential chemical reactions possible in UV illuminated gases. Zenty describes the use of UV over the wide spectral range of 750 nm to 150 nm with his description focusing on the impact of UV in the range of 240 nm to 400 nm. Longer UV wavelengths are described as “making the gases photochemically reactive”. The primary impact of the UV as described in Zenty is to make exhaust gas streams more chemically reactive, reducing SO₂ and NOₓ levels by oxidation or by reaction with hydroxl radicals (OH) thereby generating useful byproducts. The disclosure of this reference is herein incorporated by reference to the extent that it is not inconsistent with this application.

U.S. Pat. No. 4,110,183 issued to Furuta et al. and entitled “Process for Denitration of Exhaust Gas” discloses using ultraviolet radiation coupled with oxygen to oxidize nitric oxide and nitrogen dioxide to acids. U.S. Pat. No. 4,416,748 issued to Stevens and entitled “Process for Reduction of the Content of SO₂ and/or NOₓ in Flue Gas” describes a method in which ammonia (NH₃) is added to the exhaust gas and then exposed to UV radiation between 170 nm and 220 nm in order to assist in the photochemical removal of the pollutants. U.S. Pat. No. 4,863,687 issued to Stevens et al. and entitled “Method for Removing. Malodorous or Toxic Gases from an Air Stream” describes using ozone combined into the organic pollutant stream and then irradiated with UV radiation in the range of 210 nm to 310 nm. U.S. Pat. No. 4,969,984 issued to Kawamura et al. and entitled “Exhaust Gas Treatment Process Using Irradiation” describes the addition of NH₃ to exhaust gas under the irradiation of UV and the method of removing the resulting dust with precipitators. U.S. Pat. No. 4,995,955 issued to Kim et al. entitled “Optically-Assisted Gas Decontamination Process” describes a method and process for using of UV with wavelengths shorter than 200 nm to remove NOₓ and SO₂ from exhaust gas streams. This method specifically invokes the generation of atomic oxygen to assist in the rapid oxidation of pollutants. U.S. Pat. No. 5,138,175 issued to Kim et al. and entitled “Lamp Sheath Assembly for Optically-Assisted Gas Decontamination Process” describes a method and apparatus for using UV below 220 nm to make exhaust gases photochemically reactive and thereby enhancing oxidation. Finally, U.S. Pat. No. 5,935,538 issued to Tabatabaie-Raissi et al. and entitled “Apparatus and Method for Photocatalytic Conditioning of Flue Gas Fly-Ash Particles” describes the use of UV radiation to generate hydroxyl (OH) radicals that reduces the SO₂ content of the exhaust gas and thereby enhances the collection efficiency of the exhaust fly ash. The disclosures of each of these references are herein incorporated by reference to the extent that they are not inconsistent with this application.

### SUMMARY OF THE INVENTION

An intense ultraviolet radiation (“UV”) source based upon a stabilized, and/or spatially focused, confined, or pulsed combustion flame (“flame”), whose thermal energy may be augmented by external sources, is disclosed which creates an intense UV output. The UV source may be, but does not have to be, segregated from the working environment by an envelope or filter. When the UV source is not segregated from the working environment, the gas by products of the UV source mix with the working environment. As used herein, an intense UV source is one that emits substantial amounts of high energy, shorter wavelength UV as well as wavelengths reaching into the visible and infrared spectrum, in comparison to typical UV sources known today. This operation allows for more efficient use of the UV that is created, as there is no containment envelope to absorb the UV. Further, the disclosed UV source may optionally comprise an electrically-augmented combustion flame, which may reduce the required amount of electrical energy input to obtain similar UV output as prior art systems and, thus; may reduce the cost of operating the UV source as well as reducing replacement costs of the UV source. The invention involves focusing, confining, or containing a combustion flame and enhancing the flame temperature with externally applied energy in such a way that the flame gases become an intense UV source.

The UV output produced according to the invention may be used for many applications including, but not limited to, the molecular dissociation of contaminants, pollutants, or combinations thereof, such as nitric oxides, nitrogen dioxides, nitrous oxides, sulfur oxides, carbon monoxide, and carbon dioxide into nitrogen, oxygen, elemental sulfur and carbon. In addition, the UV output of the UV source of the invention may be customized or enhanced to that wavelength or those wavelengths that optimally dissociate certain pollutants and/or contaminants while not affecting non-pollutants and/or non-contaminants, further increasing the efficiency of a system operated according to the invention by adding elements or compounds (impurities) to the flame gas stream in percentages typically in the range of about 0.1% to 5% by volume. Typically the UV source may be placed into an environment of an exhaust pipe carrying the pollutants and/or contaminants such that exhaust gases are substantially directly exposed to the UV. radiation emanating from the UV source. The exposure of the pollutants and/or contaminants can cause molecular dissociation of the pollutants and/or. contaminants.

It is therefore an object of the invention to efficiently produce low-cost, intense UV output, and particularly UV in the UV-C spectral band.

It is another object of the invention to efficiently produce low-cost UV output that may be used to dissociate certain molecular contaminants and/or pollutants found in the exhaust gases of combustion systems, without the need to segregate the UV source from the contaminants and/or pollutants stream to be treated. These contaminants and/or pollutants include but are not limited to nitric oxides, nitrogen dioxides, nitrous oxides, sulfur oxides, carbon monoxide, and carbon dioxide.

It is another object of the invention to efficiently produce low-cost UV output that may be used to clean up pollutants and/or contaminants in liquid effluent streams by using a gas barrier separating the UV source from the liquid.

It is yet another object of the invention to provide a system and method of producing specific UV output having a desired wavelength necessary to produce the dissociation energies for the pollutants and/or contaminants that are present in the stream to be decontaminated.

It is yet another object of the invention to produce a long-lasting intense UV source (which may be free-standing), capable of producing >2000 W per meter length, that is inexpensive to operate as compared to current UV sources (˜8-10 W per meter length).

A UV source comprises a source of a combustion flame (“flame”), which flame is passed through or around a conducting electrode that is capable of operating as a cathode (“cathode”). The cathode may be a hollow tube, needle, or mesh, preferably though not exclusively made from a refractory metal, and may be placed substantially at the end of an orifice, for example a nozzle, from which the gases generating the flame exit.

Classical (i.e., conventional) combustion generates a very slightly ionized flame. Additional energy can be input into the flame through an auxiliary electrical heating source by placing a DC or pulsed voltage source between the cathode and a second conducting electrode capable of operating as an anode (“anode”). The anode may be a hollow tube, rod, or needle, preferably though not exclusively made from a refractory metal, and may be placed substantially opposite the cathode and substantially along the axis of the combustion flame.

The auxiliary electrical heating source (“electrical input”) should be capable of causing a current to flow between the two electrodes through the initially partially ionized flame, heating the gases in the flame, further ionizing the flame thereby creating a plasma, and substantially increasing the UV output of the flame. The partial ionization of the flame produced by combustion allows lower voltage operation of the auxiliary electrical heating source. The heating of the invention is in the form of an electrical discharge (“electrical discharge”) where the flame plasma is formed by injected or ambient gases. Lower levels of electrode erosion are observed with heating of the flame by an electrical discharge than previously seen in other types of plasma formation. The electrical discharge and the flame are substantially spatially identical. The electrical input into the flame can be adjusted to produce a desired intensity of UV output from the source (“UV output”).

A combustion flame through which an electrical current flows can also be spatially confined, contained, or focused in a number of ways. For example, a substantially symmetric conducting grid, (the “focusing grid”) can be placed around the flame in order to apply a substantially radial electric field to the electrical discharge. The geometry and the magnitude of the electric field can be modified or tailored to produce the desired axial profile of the flame. The flame axis can be substantially located along the axis of the focusing grid. In a preferred embodiment, the focusing grid is symmetric to the flame, such as a cylinder; however other shapes may be used.

Alternatively or additionally, magnetic fields can be used to spatially confine the flame. Permanent magnets or electromagnets can be placed such that the magnetic field is aligned substantially along the axis of the flame. Alternatively or additionally, concentric shells of gas flowing from a concentric annular nozzle can be used to contain the combustion flame into a well-defined extended-length cylinder by balancing the gas pressure generated by the flame with the pressure of the surrounding gas jet. This effect can be seen prior to the establishment of an electrical discharge into the flame but will continue to confine the hot gases following the start of the electrical discharge. Finally, a compression wave emanating from a pulsed detonation of non-burning combustion gases initiated by a pulsed electrical discharge can be used to compress or confine the hot gases or the plasma created by an axial flame. These confinement techniques can be used separately or together to optimize the UV output from the flame plasma.

The source of the UV output according to the invention does not have to be physically segregated from the working environment, thus allowing it to be placed within any environment that is to receive the UV. The lack of a material envelope increases the intensity of the UV output seen by, and applied to, the local environment and, particularly, increases the presence of shorter wavelength UV in the UV output. The presence of shorter wavelength UV radiation allows a higher efficiency for molecular dissociation.

The UV output may thus be varied by several techniques including changing the combustion parameters to alter the composition or shape of the flame, increase the temperature of the flame, alter the magnitude of the electrical input through the flame, or minimize the flame volume using confinement techniques. Electromagnetic radiation sources, such as radio-frequency induction heaters, microwave generators, or lasers, may optionally be used to augment the heating of the flame plasma following heating from combustion or the electrical discharge. For pulsed operation, the gas and electrode temperatures should be maintained below the ignition temperature of the combustion gas.

Gas constituents adjacent to or in the flame may be modified by the addition of one or more impurities to enhance UV production in specific spectral bands, as desired according to the molecular dissociation energy requirements of various contaminants and/or pollutants. The efficiency of the molecular dissociation may be even further improved by tailoring a UV spectral output so that it is minimally absorbed by non-pollutant species.

The UV source may be operated so that the combustion flame emits about 500 to about 2,000 or more, preferably from about 1,000 to about 2,000 W per meter length of the combustion flame.

Finally, the overall efficiency of the UV source may be improved by incorporating reflecting walls into the system to return the infrared and visible portion of the emitted spectrum back to the flame, further increasing the energy input into the flame.

### BRIEF DESCRIPTION OF THE DRAWINGS

The invention will be described with reference to the accompanying drawings in which like elements are referenced with like numerals.

FIG. 1a is a schematic depicting a nozzle and rod electrode configuration with a propane flame having an electrostatic force that is parallel and has the same direction as the thermal buoyancy of the flame.

FIG. 1b is a schematic of the embodiment of the system of FIG. 1a showing the rotation of the electrical discharge around the cooler portion of the flame and the anode.

FIG. 2a is a schematic showing a nozzle and rod electrode configuration with a propane flame having an electrostatic force that is parallel but in the opposite direction than the thermal buoyancy of the flame.

FIG. 2b is a schematic of the embodiment of FIG. 2a showing the rotation of the electrical discharge around the cooler portion of the flame.

FIG. 3a is a schematic of a propane flame flowing through screen electrodes.

FIG. 3b is a schematic of the embodiment of FIG. 3a showing the location of the electrical discharge between the screen electrodes.

FIG. 4a is a schematic of a nozzle and rod electrode configuration with a propane flame having a cylindrical electrostatic focusing grid biased negatively with respect to ground potential.

FIG. 4b is a schematic of the embodiment of FIG. 4a showing the electrical discharge through the core of the flame without rotation.

FIG. 4c is a schematic of the embodiment of FIG. 4a showing the flame shape when the electrostatic force generated by the focusing grid exceeds the thermal buoyancy force of the flame.

FIG. 4d is a schematic of the embodiment of FIG. 4c showing the path of the electrical discharge when the electrostatic force is larger than the buoyancy force.

FIG. 5a is a schematic showing a nozzle and rod electrode configuration with a propane flame having an electrostatic force that is parallel to and has the same direction as the thermal buoyancy of the flame, with installed magnets.

FIG. 5b is a schematic of the embodiment of FIG. 5a showing the rotation of the electrical discharge around the cooler portion of the flame.

FIG. 6a is a schematic showing a nozzle and rod electrode configuration with a propane flame having an electrostatic force is parallel to and the same sign as the thermal buoyancy of the flame, and with the flame compressed with an annular gas jet.

FIG. 6b is a schematic of the embodiment of FIG. 6a showing the electrical discharge passing through the core of the flame.

FIG. 7 is a schematic of various suitable cathode configurations.

FIG. 7a depicts a blunt rod cathode.

FIG. 7b depicts a sharpened rod cathode.

FIG. 7c depicts a blunt rod cathode with a sharp, doped-tungsten or iridium tip.

FIG. 7d depicts a tubular cathode with a sharp refractory tip comprising tungsten or iridium.

FIG. 7e depicts a tubular cathode.

FIG. 7f shows a blunt rod cathode with installed magnets.

FIG. 8 is a schematic of various anode configurations.

FIG. 8a depicts a blunt rod anode.

FIG. 8b depicts a sharpened rod anode.

FIG. 8c depicts a tubular anode.

FIG. 8d depicts a blunt rod anode with installed magnets.

FIG. 9a is a schematic showing a nozzle and rod electrode configuration with a propane flame having an electrostatic force that is parallel to and having the same direction as the thermal buoyancy force of the flame with a pulsed electrical input.

FIG. 9b is a schematic of the embodiment of FIG. 9a showing the electrical discharge centered in the core of the flame.

FIG. 10 is an ultraviolet and visible light spectrum of the output of the embodiment of

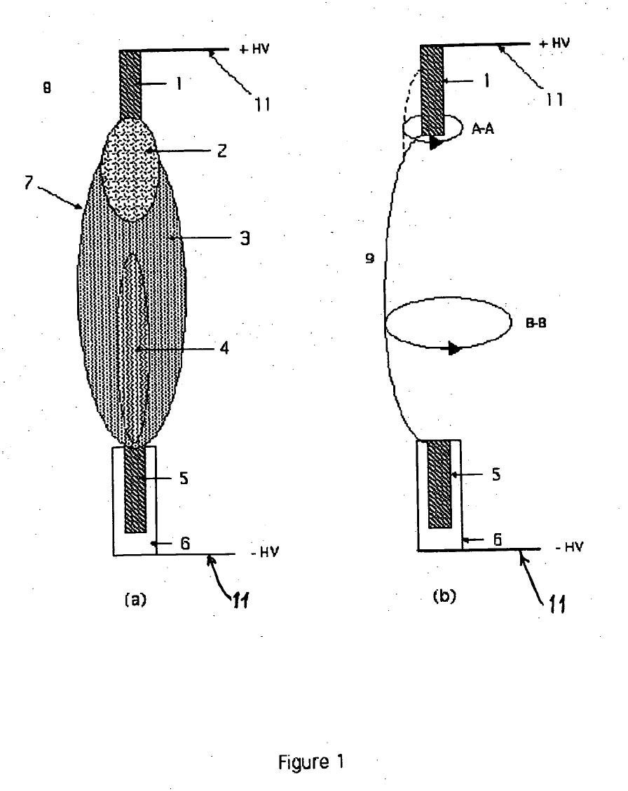

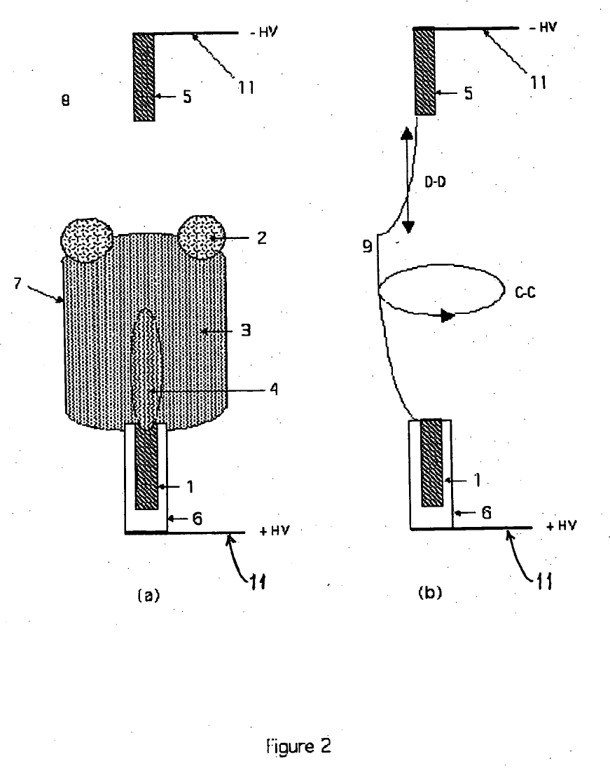

FIG. 1a while using-a DC electrical input and a magnetic field along the flame. The flame combustion used a fuel/air mixture having air in excess of that needed for stoichiometric combustion (“lean mixture”).

FIG. 11 is an ultraviolet and visible light spectrum of the output of the embodiment of FIG. 1a while using a DC electrical input and a magnetic field along the axis of the flame. The flame combustion used a fuel/air mixture having fuel in excess of that needed for stoichiometric combustion (“rich mixture”).

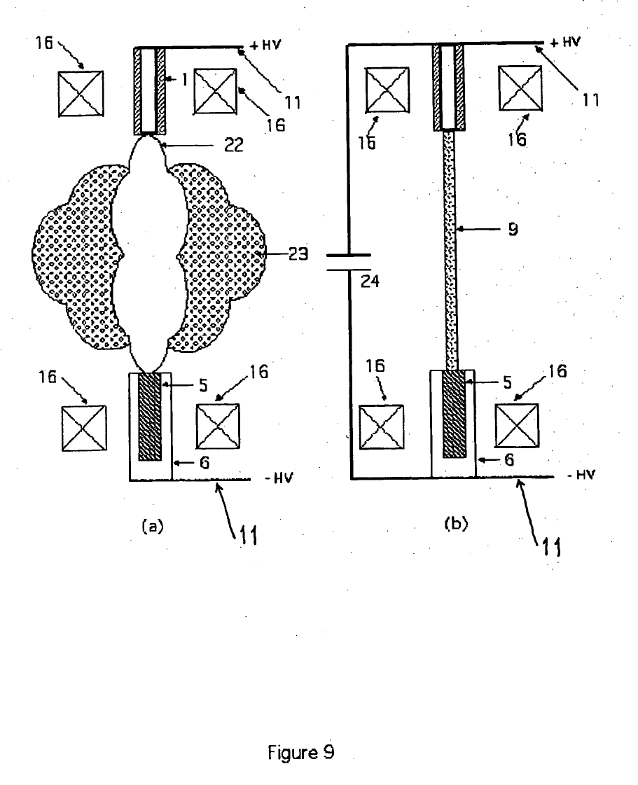

FIG. 12 is an ultraviolet and visible light spectrum of the output of the embodiment of FIG. 9a while using a pulsed electrical input and argon injected from the anode, with a 1.0 μF capacitance at a 10 kV electrical input.

FIG. 13 is a schematic of the embodiment of FIG. 9a applied to the application of treating exhaust gas from a combustion source showing the UV source located in an exhaust pipe.

FIG. 14 is a schematic of a UV source showing a nozzle and rod electrode configuration with a propane flame surrounded by an auxiliary source of energy for the electrical discharge, in this case a radio-frequency induction heating coil.

### DETAILED DESCRIPTION OF THE INVENTION

A free-standing, intense UV source according to the invention includes a source of a combustion flame, an orifice, such as a nozzle, through which the combustion flame exits, a conducting mesh (or grid) or other electrode capable of operating as a cathode, preferably though not exclusively made from refractory metals such as tungsten or iridium, placed in the vicinity of the exit of the orifice, preferably immediately after the exit of the orifice, a conducting needle, rod, or tube (or other electrode shape) capable of operating as an anode, preferably though not exclusively made from a refractory metal such as tungsten or iridium, placed substantially in the axis of the flame, an electrical discharge input into the flame, and a flame or electrical-discharge confinement element.

The combustion fuel may include many different fuels, such as diesel fuel, hydrogen, acetylene, propane, butane, or methane, or mixtures thereof. The amount of fuel needed is determined by the size of the system but may be as low as about 1 to about 3 cm³.min.

An oxidizer, such as oxygen, fluorine, chlorine, or air, is required to generate the combustion flame. The oxidizer amount is determined by the need for near (or substantially) stoichiometric combustion. The fuel selection may be influenced by the final UV spectrum desired from the system. The combustion fuels are typically combined with the oxidizer and passed through a nozzle capable of withstanding the temperatures generated where they are burned yielding a combustion power typically in the range of about 0.1 kW to about 10 kW. Larger combustion power outputs are possible but they add little to the overall UV performance of the system. This combustion power is calculated by knowing the fuel flow rate and the heat content of the fuel. The combustion results in a high temperature flame, ranging from about 1000° C. (about 1800° F.) to about 2900° C. (about 5000° F.), depending upon the operating conditions, fuels, and oxidizers. Those skilled in the art will be capable of constructing such a system without undue experimentation using the guidelines provided herein.

In one embodiment, the flame surrounds the cathode or may pass through a conducting mesh acting as a cathode, which preferably is connected to the nozzle and is typically at ground potential. The anode may be placed a distance from the conducting mesh or electrode at the nozzle, preferably about 1 cm to about 10 cm and most preferably about 5 cm from the conducting mesh or electrode. The polarity of the anode may be negative with respect to ground. A voltage of about 1 kV to about 20 kV, and preferably about 10 kV, is applied to the cathode from a DC or pulsed voltage source placed at a suitable location, such as between the anode and the cathode, causing an average current of at least 0.1 A to about 10 A to pass through the flame, further ionizing and heating the already partially ionized hot flame with an additional electrical power from about 100 W to about 20 kW. Typically, the average electrical power added by the electrical input is comparable to or up to 10 times the power from the combustion process. In the case of a pulsed current the peak current may reach 5,000 A or more. Input electrical power can easily be determined by those skilled in the art according to the desired intensity and spectral brightness of the desired UV output of the system. The energy delivered to the combustion flame by the electrical discharge may further be augmented by an external electromagnetic radiation source, such as a laser, a microwave generator, or a radio-frequency induction heater, focused on the flame. Those skilled in the art will be capable of designing such a system without undue experimentation using the guidelines provided herein.

Without wishing to be confined to any theory of operability, the combustion flame and associated electrical discharge can be contained or confined by the use of an annular gas jet surrounding the combustion flame. The annular gas jet can be created using an annular nozzle that surrounds the combustion nozzle. This nozzle can be sonic or supersonic depending on the needed containment parameters. Choosing a volumetric flow rate comparable or several times larger than the total flow rates of the combustion gases allows the combustion gases to be partially or totally contained thereby extending the length and uniformity of the flame. The containment gases used can be any readily available gas. Air, nitrogen, or oxygen-depleted combustion gases work well.

Additionally, a magnetic field generated by permanent magnets or electromagnets can be used to confine or contain (i.e., stabilize) the flame and the electrical discharge. The ionization level provided by the electrical discharge enables the magnetic field to have an appreciable effect. The magnetic field should be at least 500 Gauss but improvements in confinement are seen with increasing magnetic field. The limit of the magnetic field is determined by practical limitations associated by the generation techniques. For example, permanent magnets have peak magnetic fields of ˜5 kG. The magnetic field is generally aligned with the axis of the flame. Those skilled in the art will be able to design confining magnetic field geometries without undue experimentation using the guidelines provided herein.

Additionally, an electrostatic focusing grid can be used to stabilize, confine, and focus the flame that contains the electrical discharge, compressing the flame into a smaller volume than would otherwise be the case and increasing the peak flame temperature that, in turn, increases the flame plasma density and the UV output of the system. The focusing technique along with an optional electromagnetic radiation input into the flame is capable of intensifying the UV output of even a relatively modest UV source. The focusing grid is preferably symmetric around the axis of the combustion flame, most preferably a cylinder, but other shapes (such as slots) are possible and can be modified to produce the desired flame axial (or two dimensional) profile. The focusing grid should be biased negatively with respect to ground, preferably with a potential between about 1 kV and about 15 kV, most preferably about 10 kV. The electrical current at the focusing grid should be nearly zero as in normal operation the grid should draw no current. Typical grid leakage currents preferably are less than about 10 microamperes. Those skilled in the art will be able to design a focusing grid without undue experimentation using the guidelines provided herein.

The output UV power (as measured by a calibrated UV photometer) and the UV spectrum (as measured by a UV spectrometer) of the UV source can be varied in several different ways, according to the specific application. Average output power in the UV-C spectrum of greater than 10 W from a 2-cm long electrical discharge (distance between electrodes) can be observed. For example, the choice of fuels (or fuel/oxidizer ratios) can alter the flame temperature and flame elemental composition, which in turn alters the UV output from the flame. Increasing the magnitude of the electrical input through the flame or adding electromagnetic radiation input into the flame can increase the flame temperature. Also, the addition of impurities, such as but not limited to helium, neon, argon, and xenon, to the combustion fuel and/or oxidizer, typically at about 0.1% to 5% by volume, can alter the output UV spectrum. Liquids containing impurities such as salts can be used to replace expensive gases in order to place impurities in the flame. Examples of suitable impurities include but are not limited to boric acid, potassium chloride, sodium chloride and lithium hydride. The amount of added impurities can be adjusted as necessary to achieve the desired UV spectrum for the application. The desired UV spectral output may be determined by inspection of the molecular dissociation energies of the pollutants and/or other contaminants in the environment to be treated, from sources well known in the art, and the selection of an UV wavelength that will dissociate the pollutants and/or other contaminants yet not be absorbed by other pollutants and/or other contaminants.

The intense UV source may be used to treat waste streams containing pollutants and/or contaminants. For example, combustion system gas exhaust or any other waste stream may be treated by exposure to intense UV to cause molecular dissociation and/or chemical reaction of the pollutants and/or contaminants in that waste stream. The UV source may be segregated from the working environment if necessary or desirable, depending upon the application. However, the UV source may also be exposed directly to the working environment with no barrier between the UV source and the working environment, such as an envelope or other barrier that may act as a filter.

Turning now to the drawings, FIG. 1 shows one embodiment of the invention comprising blunt rod-like electrodes for the anode 1 and the cathode 5, and a nozzle 6 injecting a propane flame 7. The flame 7 may comprise a hot light blue region 4 near the nozzle 6, a darker region 3 surrounding the light blue region 4, and a white colored region 2 near the anode 1. The anode 1 and the cathode 5 are located substantially opposite of each other in a configuration such that the electrostatic force acting on the flame 7 formed by an electric field between the anode 1 and the cathode 5 formed due to electrical input 11 into the flame 7 is substantially parallel with and adds to the thermal buoyant force of the flame 7. In this configuration, the fuel-oxidizer ratio, electrode to flame height, and the magnitude of the current between the anode 1 and the cathode 5 can be varied. The current between the anode 1 and the cathode 5 may be continuous or pulsed.

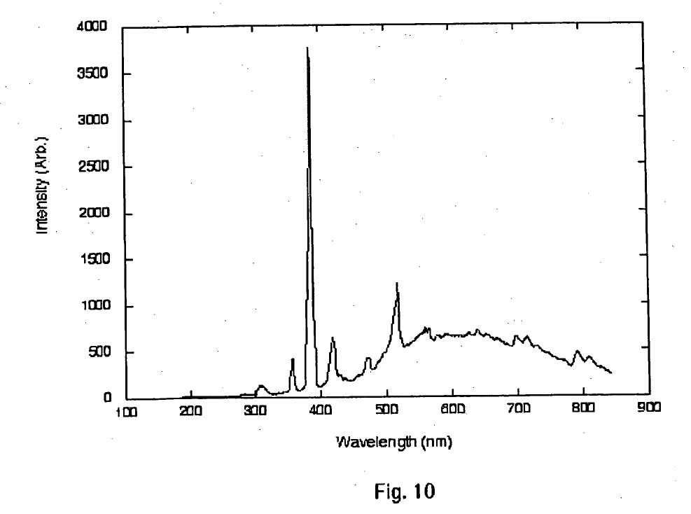

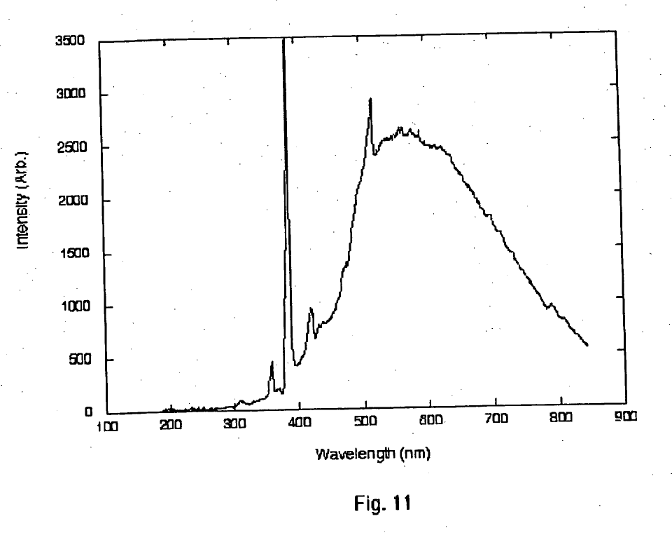

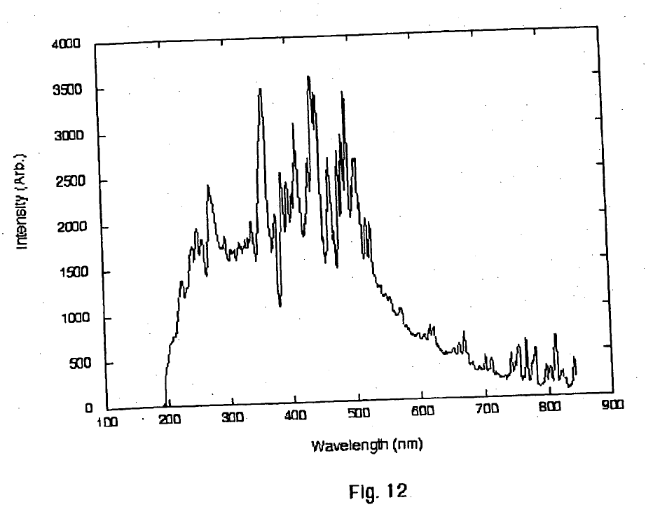

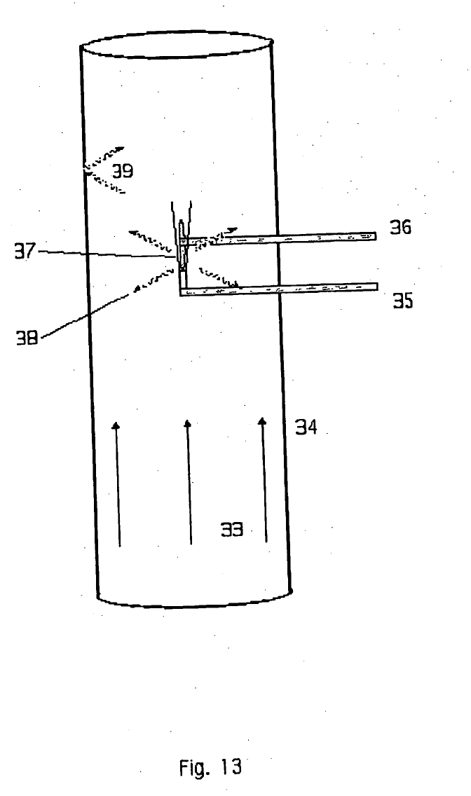

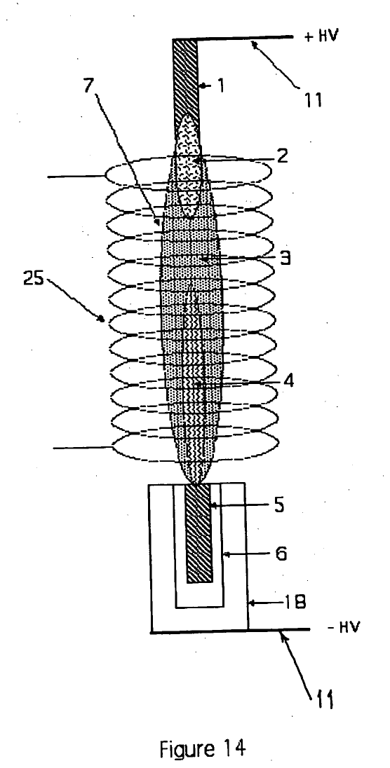

For lean flames (flames with excess oxidizer), evidenced by a flame ranging from a light blue to clear color, the UV output of the system can produce line emissions identified coming from C—H, O—H, C—N, and C—O bonds between 300 nm and 550 nm and a small Planckian profile with a peak at 600 nm as shown in FIG. 10. These data were taken with an ultraviolet grating spectrometer (Stellarnet Inc. EPP2000C™) fiber-optically coupled to a collecting optic located ˜1 m from the UV source. For rich flames (flames with excess fuel), evidenced by a flame color ranging from light blue with a white tip to an all white, the UV output of the system can produce line emissions identified to be from C—H, O—H, C—N and C—O bonds between 300 nm and 530 nm and a large, visible-light Planckian profile as shown in FIG. 11. These data were measured as described above.

For the design of electrodes depicted in the embodiment of FIGS. 1a, the electrical discharge 9 of the system typically follows the dark blue interface substantially located between the flame 7 and external air 8 as seen in FIG. 1b. The electrical discharge 9 typically moves erratically around the anode 1 in a pattern as noted by the line marked A-A and the flame 7 in a pattern as noted by the line marked B-B. For a cathode 5 with a thin rod electrode (not shown), the electrical discharge 9 will typically travel substantially through the center of the flame 7 and adding to the stability and reproducibility of the discharge 9.

The overall electrical current/voltage (impedance) behavior of the source configuration of FIG. 1a where the anode 1—cathode 5 (“electrode”) gap was ˜2 cm varies with electrical input. At an electrical input current 11 of about 25 mA, the average electric field strength between the electrodes in one configuration similar to that of FIG. 1a was 6-8 kV/cm; at a current 11 of 50 mA, the average electric field strength between the electrodes of the same configuration was 4-6 kV/cm; at a current 11 of 100 mA the average electric field strength between the electrodes of the same configuration was 2-4 kV/cm; and at a current 11 of 200 mA, the electric field strength of the same configuration was 1-2 kV/cm. The electric field was determined by measuring the voltage across the electrodes and the electrode gap. Thus, the impedance of the discharge is seen to decrease with increasing voltage. Without wishing to be bound by any theory of operability, this is believed to be due to the increasing plasma ionization level and average plasma temperature in the flame. These data imply that a high amperage pulse, perhaps generated through capacitive discharge, could be capable of dropping the voltage required to sustain an electrical discharge to ˜100's of volts.

FIG. 2 depicts another embodiment comprising rod-like electrodes for the anode 1 and the cathode 5 and a propane flame 7 injected through a nozzle 6. The anode 1 and the cathode 5 are again located substantially opposite each other, but are oriented in this embodiment such that the electrostatic force on the flame 7 formed by the electrical field between the anode 1 and the cathode 5 is substantially parallel to but opposite the thermal buoyant force of the flame 7. The overall shape of the flame 7 is typically markedly different in this configuration than the embodiment shown in FIG., la. The flame 7 is typically compressed toward the nozzle 6 with a light blue region 4 near the nozzle and a flattened darker blue region 3 substantially around and above the nozzle 6. Typically a bright white spot 2 is noticed substantially between the edge of the dark blue region 3 and the cathode 5.

For low electric fields between the anode 1 and the cathode 5 (lower voltages or larger anode-cathode gaps), the flame 7 may change position between the anode 5 and the cathode 1 and can create large vortices at the flame tip 2. For high electric fields between the anode 1 and the cathode 5 (higher voltages or smaller anode-cathode gaps), the flame 7 may burn nearly horizontally in relation to the anode 5 and the cathode 1. This flame configuration fails to provide a continuous ionization path in the anode-cathode gap. Turning to FIG. 2b, the electrical discharge 9 according to this embodiment may rotate erratically around the dark blue region 3 in a pattern shown by the line marked C-C and may fluctuate continuously between the anode 1 the cathode 5 in a manner as depicted by the line marked D-D.

For lean flames (flames with excess oxidizer), evidenced by a flame 7 ranging from a light blue color to a clear color, the UV output of the system shown in FIG. 2a typically produces line emissions identified to be from C—H, O—H, C—N, and C—O bonds between 300 nm and 550 nm and a small Planckian profile with a peak at 600 nm as shown in FIG. 10. For rich flames (flames with excess fuel), evidenced by a flame color ranging from light blue with a white tip to all white, the UV output of the system typically produces line emissions identified to be from C—H, O—H, C—N and C—O bonds between 300 nm and 530 nm and a large, visible-light Planckian profile as shown in FIG. 11. One disadvantage for rod electrode designs in the embodiments depicted in FIGS. 1a and 2ais that, at the DC current electrical input levels 11 needed for desirable UV-C output, hard carbon can build up on the cathode 5 and sooty carbon can build up on the anode 1 until the gap between the anode 5 and cathode 1 is bridged. The anode 5 can require external cooling to prevent melting at high current levels.

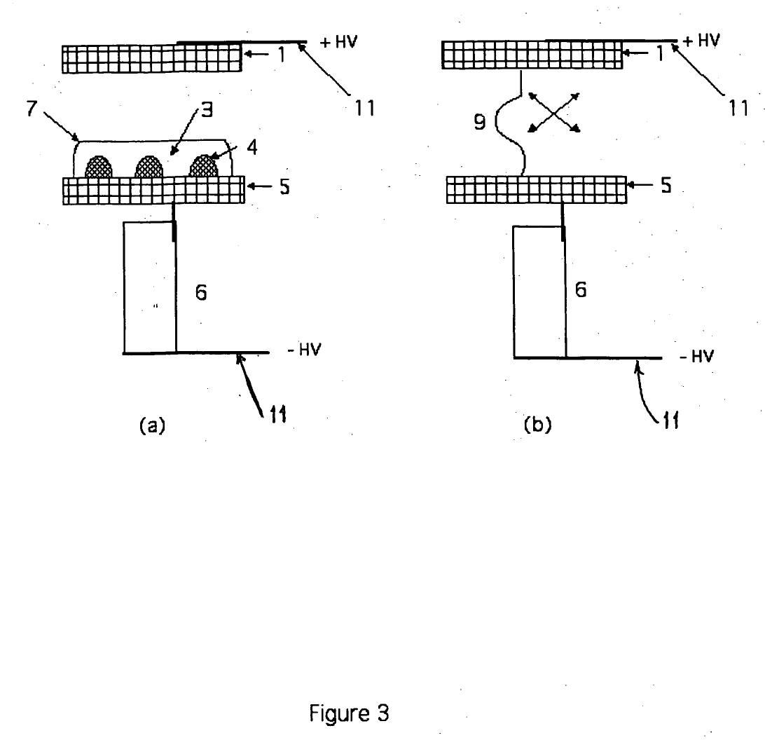

FIG. 3a depicts an embodiment comprising a screen or slots for both of the anode 1 and the cathode 5. The flame 7 typically burns stably on the screen or slots 1, 5 similar to a Bunsen burner. There may be multiple light blue regions 4 and darker blue regions 3 where the fuel, such as propane, flows through the screen or slots 5 from a nozzle 6.

As seen in FIG. 3b, for a low current electrical input 11, the electrical discharge 9 may vary its position on and around the screen or slots 5, and may also find the path of least resistance through the screen or slots 5. For high current electrical input 11, the electrical discharge 9 can melt the thin wires that make up the screen 5 or the electrical discharge 9 can heat up the thick wire that makes up the slots 5. If the electrical input voltage is too low, there may be insufficient voltage to establish and maintain an electrical discharge. The damage threshold of the screen or slots sets the upper limit to the electrical input current 11. The UV-C output of the system is typically much lower in the embodiment with the screen-stabilized flame 7 as compared to the embodiments of FIGS. 1a and 2a, but typically very little carbon build up is found on the anode 1 or cathode 5 most likely because the discharge 9 does not maintain a constant position on the screens 1 and 5 but rather is constantly moving around these surfaces.

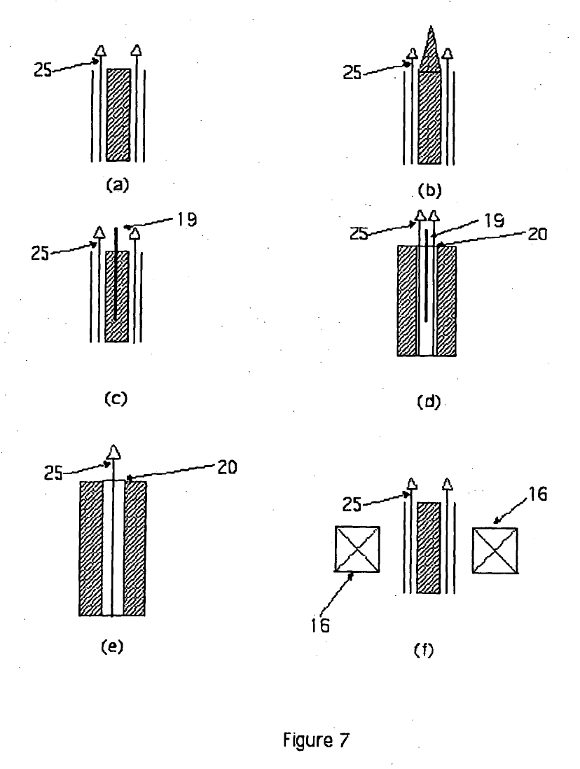

FIG. 7 depicts a selection of cathode electrode designs (not including screens or slots) that are suitable for use in the invention, although any cathode design now known or later developed can be used. The suitability of a cathode design is primarily related to the lifetime of design followed by the reproducibility of the electrical discharge. The depicted cathode designs are a blunt rod electrode (FIG. 7a); a sharp rod electrode (FIG. 7b); a conductive rod with a refractory or high-temperature material tip 19 such as tungsten or iridium electrode (FIG. 7c), a hollow rod or tubular electrode (FIG. 7e); and a hollow rod (tube) electrode with a refractory or high-temperature material-tip 19 such as tungsten or iridium (FIG. 7d).

The combustion gas 25 flows around or through the cathode electrode. The combustion gas 25 may contain impurity gases designed to enhance the UV output or modify the UV spectrum, typically at about 0.1% to 5% by volume. The blunt tip cathode depicted in FIG. 7a does not typically encourage a stable electrical discharge path, which can lead to unstable UV-C output in the system. The sharp rod cathode of FIG. 7b exhibits desirable UV-C emissions in the UV output, but the tip of the sharp rod can build up carbon if exposed to the flame. Additionally, the sharp tip may erode over time becoming blunt at higher discharge current levels or by a choice of a low-melting-point metal for the electrode. If the sharp rod is made of refractory metal such as iridium it typically will last for many hours of continuous operation.

Similar to the screen cathode depicted in the embodiment of FIG. 3a, the tube electrode of FIG. 7e typically does not build up carbon likely due to the gas flowing past the tube electrode. Argon and/or hydrogen can be input as additives to the combustion gas in the gas jet to enhance UV-C generation in such embodiments. The tube cathode can sustain high current electrical input loads because the electrical discharge of the system rotates on the rim of the tube 20.

Yet another cathode design as shown in FIG. 7d combines the hollow tube 20 of FIG. 7e with a thin, sharp rod 19. During the initial electrical discharge of the system, current begins to emanate from the tip of the sharp rod 19. As the electrical discharge current increases, the thin rod 19 typically heats up, thus becoming more resistive. The electrical discharge then typically will anchor onto the tube rim 20 as the electrical discharge current increases. The cathode life can be extended for high system discharge currents by using a thin rod electrode 19 and hollow rod or tube combination electrode as a cathode.

Magnetic stabilization of the flame can be realized as seen in the embodiment of FIG. 7f by placing a magnetic field using magnetic sources 16 substantially along the axis of the flame. When magnetic stabilization is combined with the cathode of FIG. 7d, the electric discharge of the system tends to start on the tip of the thin rod cathode 19 and transfer to the electrode then to rotate on the tube rim 20. Those skilled in the art will be able to design suitable cathodes without undue experimentation using the guidelines provided herein.

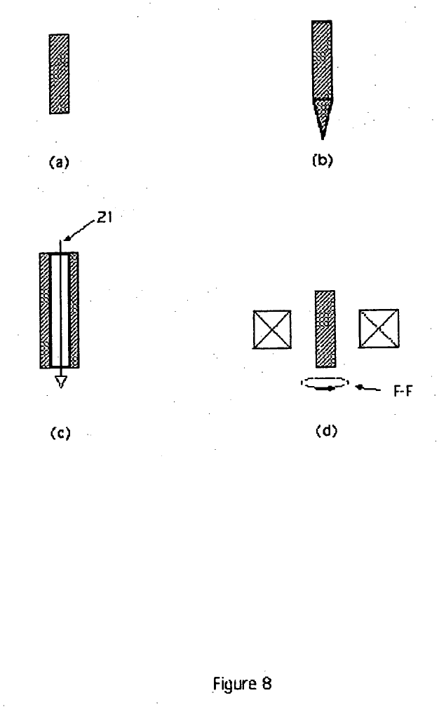

FIG. 8 depicts several anode designs suitable for use in the invention, although any anode design now known or later developed may be used. The suitability of an anode design is primarily related to the lifetime of design followed by the reproducibility of the electrical discharge. The depicted anode designs are a blunt rod electrode (FIG. 8a), a sharp rod electrode (FIG. 8b), and a hollow rod electrode (FIG. 8c). For low system electrical discharge current, all of the depicted anode designs performed substantially the same. For high system electrical discharge current, the blunt tip of FIG. 8a can quickly become a sharp tip as shown in FIG. 8b as metal melts and is deposited on the cathode. To prevent such melting, the tubular anode design of FIG. 8c with a gas cooling flow 21 can be used. As the electrical discharge system current load increases, the cooling of the anode tube should increase to prevent melting/wearing of the tip of the anode tube. Magnetic stabilization (FIG. 8d) can be used to encourage the uniform and reproducible electric discharge and to rotate the discharge on the rim of a solid rod or hollow tube in a pattern depicted by the line F-F.

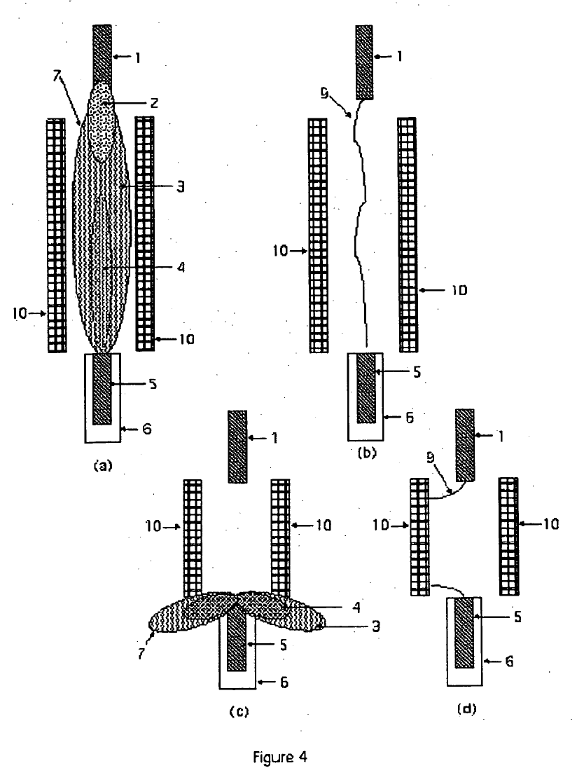

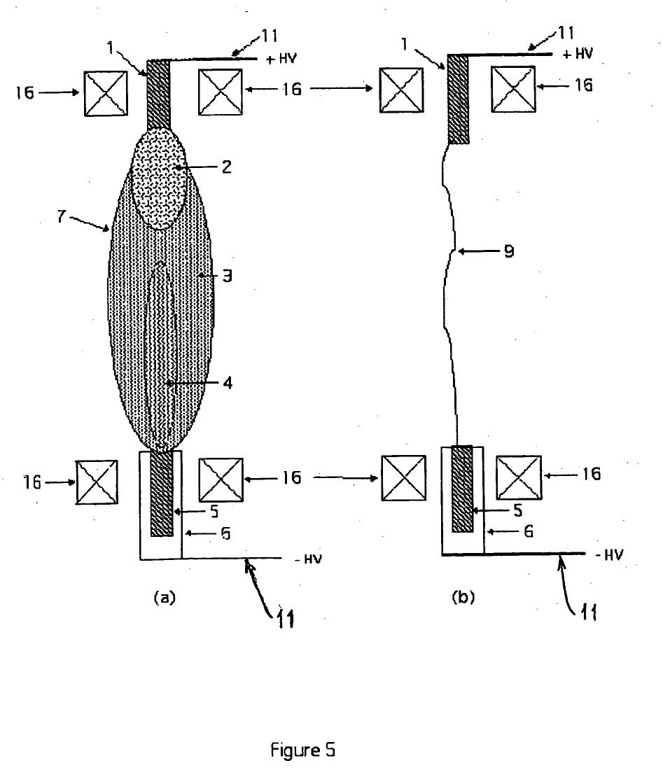

To increase the UV-C output of the system, any number of discharge compression methods can be utilized. Several methods are shown in FIGS. 4, 5, and 6; respectively, electrostatic force, magnetic force and gas shear force. FIG. 4a depicts the use of electrostatic force that can be created, for example, by charging a conducting screen 10 such as copper, brass, or stainless steel, or a conducting quartz or sapphire tube 10. A flame 7 can be created that exits from a nozzle 6. FIG. 4b shows a DC current discharge 9 will flow between the cathode rod electrode 5 and the anode rod electrode 1 as the electrical discharge. Up to about 10% increase in UV-C output could be measured using this technique of electrostatic compression over a similar system with no electrostatic compression.

FIG. 4c demonstrates that, if the electrostatic force exceeds the thermal buoyant force of the flame, the flame 7 can burn horizontally between the anode 1 and the cathode 5, or even upside down with its tip substantially extended in the direction of the nozzle 6 rather than extending away from the nozzle 6 as in other embodiments. The electric discharge 9 of the system, in this instance, can short through the screen or tube 10 to the cathode 5 and anode 1, as seen in FIG. 4d. When the electrostatic forces measured by voltages typically exceed 4-6 kV, the screen or tube 10 can become coated with carbon or condensed metal vapor. Even with internal and external gas jet cooled quartz and sapphire tubes, the tube 10 can heat up with high UV-C outputs, causing the electrical discharge of the system to short on the screen or tube 10 causing screen or tube failure.

FIG. 5 demonstrates the method of magnetic compression of the discharge 9. The depicted embodiment comprises a high temperature nozzle 6, a rod electrode cathode 5, a rod electrode anode 1, and one or more permanent (or electro) magnets 16 located near the cathode 5. One or more magnets 16 may also be placed at the anode of FIG. 5, as shown in FIG. 8d, in order to produce a more uniform magnetic field across the discharge 9. The magnetic field should be as large as is practicable. A flame 7 can be formed using a desired fuel mixture having a light blue region 4, a dark blue region 3, and a white colored region 2 substantially near the anode 1. A DC discharge 9 typically will be generated between the cathode 5 and the anode 1. Low levels of magnetic field placed along the axis of the nozzle 6 can also stabilize the electrical discharge 9 between the electrodes. High magnetic fields stabilize and compress the discharge as desired, but at some point permanent magnets fail to provide magnetic fields of such magnitude or electromagnets become bulky and consume large amounts of electrical power.

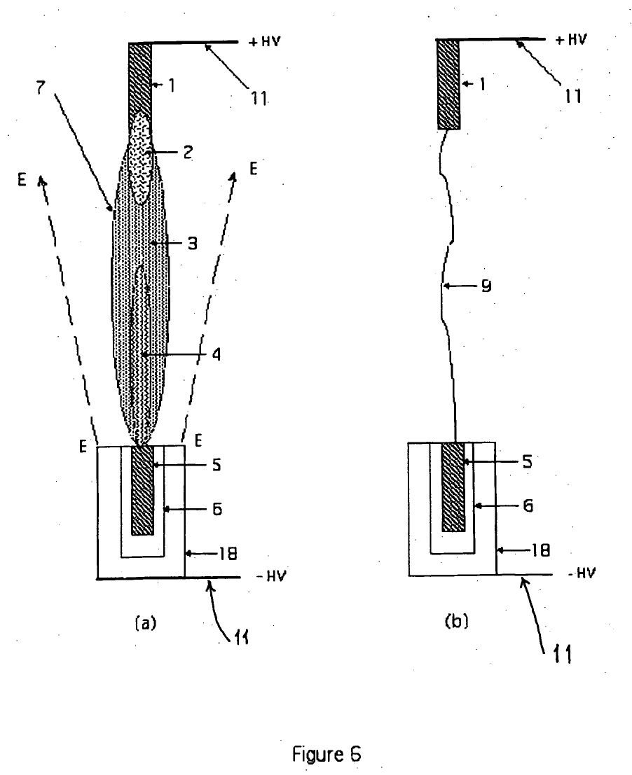

In the embodiment shown in FIG. 6, a high temperature nozzle 6 used to inject the combustion gases can be placed inside an annular gas jet 18 to confine and compress the flame 7 generated by said combustion gases. The flame 7 typically will extend between the anode 1 and the cathode 5. The flame 7 typically will be compressed and extended by the addition of a gas from the annular gas jet whose outer edge flows along the lines depicted by the letters E-E. The flame 7 may have a light blue colored region 4 nearest the nozzle 6, a dark blue colored region 3 surrounding the light blue region 4 and a white region 2 substantially in contact with the anode 1. The electrical discharge 9 is typically confined to the core of the flame 7 but may be erratic at low discharge current levels necessitating a slight increase in electrical input voltage. For annular gas flow rates substantially equal to or slightly greater than the overall flow rates of the gases feeding the flame 7, the length of the electrical discharge 9 can be extended for a fixed voltage and a fixed fuel rate using this compression technique. The external annular gas jet helps to focus the flame 7 towards the anode 1. For annular gas flow rates higher than the overall flow rates of the gases feeding the flame, 3-6% hydrogen gas maybe necessary to be added to the fuel of the combustion gas to increase the flame speed. High annular gas flow typically does not continue to increase the length of the electrical discharge 9 as compared to the low annular gas flow rates using this compression technique. Suitable gases for the external annular gas jet include air, nitrogen or oxygen-depleted combustion gases.

Using each of the compression techniques of the flame 7, up to a 10% increase in UV-C output of the system can be obtained as compared to similar flame and electrode configurations operating with no compression techniques. Compression techniques may be combined. The source configurations shown in FIGS. 1, 4, 5, & 6 having rod like electrodes have been found to radiate desirable UV-C output per unit of total energy input. However, rod electrodes experience carbon and heat build-up at high currents as previously discussed.

FIG. 9 depicts a pulsed ignition/discharge UV-C flame source system in which a capacitor 24 is placed between the anode 1 and the cathode 5. All elements of the high-voltage circuit remain the same. The desirable configurations for producing good UV output in pulsed configurations are typically the same as desirable configurations for producing good UV output found with DC operation. The system of the depicted embodiment comprises a high temperature nozzle 6 injecting a fuel and/or oxidizer mixture between a cathode 5 and an anode 1. This cathode configuration is similar to that described in FIG. 7c. A high voltage capacitor 24 can be directly connected between the anode 1 and the cathode 5 in such a way as to minimize the total resistance and inductance of the electrical discharge circuit of the system. An electrical switching element does not have to be included between the capacitor and the UV source. Switching of the capacitor is via “self breakdown” through the gases between the electrodes and is very reproducible. As the high voltage power supply charges up the capacitor, a voltage is reached where a break down occurs between the anode 1 and the cathode 5. The “self breakdown” will occur every time the capacitor is recharged by the high voltage power supply.

Depending on the current capacity of the power supply and the capacitance of the capacitor, the repetition rate of this breakdown can be high. For example, a 3-μF capacitor and a 200 mA power supply is observed to have a repetition rate of 3-5 Hz. During system electrical discharge, the discharge 9 is almost substantially aligned to the axis of the flame 7 . The flame region 22 surrounding electrical discharge 9 is very bright in the visible spectrum. Finally, a larger expanding flame region 23 may be observed expanding outward from the axis of the discharge by 3-5 cm.

For slightly rich fuel mixtures (flames with excess fuel), the Planckian peak is moved from 600 nm to 500 nm for a 0.33-μF capacitance at 5-kV electrical input to 400 nm (See FIG. 12.) for a 1.0-μF capacitance at 10-kV electrical input, and finally to 250 nm for a 10-μF capacitance at 18-kV electrical input. Doubling the electric input energy can double the UV-C output at such current loads. Operation with a 10-μF capacitor can greatly increase thin rod electrode erosion rate, but thick copper or brass tube electrodes with gas cooling placed on the anode 1 and the cathode 5 are not damaged significantly. Magnetic fields added to the pulsed configuration using permanent magnets 16 placed around the anode and the cathode have been observed to increase the UV output by ˜10%.

One embodiment of the UV source shown in FIG. 9 comprises an iridium tip on the cathode 5 and copper or brass in the nozzle 6, similar to the configuration described in FIG. 7c. The cathode 5 comprises copper and brass, and, preferably, is cooled by the combustion air jet surrounding it. The anode 1 may use a tube configuration as seen in FIG. 8c that is cooled by gas flow. One mode of operation adds argon as a UV enhancing impurity through the anode tube 21. The preferred fuel for the depicted embodiment is propane and the preferred oxidizer is oxygen, although air will provide satisfactory system performance.

There are two basic combustion modes of operation: the combustion flame can burn continuously or the fuel burns only when detonated by the pulsed electrical discharge. Detonation of the fuel gas by the pulsed electrical discharge is observed to greatly increase UV-C output, yet while the flame burns continuously, UV-C output can drop to 20% of the pulse combustion value. Average UV-C power output of >10 W has been observed with operation of the system in the pulsed mode described above. The measurement of the UV-C power was made using a calibrated photometer filtered to see only radiation in the UV-C band (UV Radiometer UVX-25™). The intensity of this UV-C power output is significantly beyond the state-of-the-art for UV sources, for example linear UV-C outputs have been observed that are more than 100× greater than currently commercially available sources.

FIG. 14 depicts an embodiment comprising a UV source with an auxiliary source of energy for the electrical discharge. Shown is a radio-frequency induction heating coil 25. Other electromagnetic radiation sources are suitable for use in the invention, including but not limited to microwave generators and lasers, which may also be used to augment the heating of the flame.

The freestanding UV source may be placed in any arbitrary gas environment where UV is desired. FIG. 13 depicts one such environment. The required combustion and impurity gases as well as the electrical energy are delivered to the UV source 37 via insulated electrodes and tubes 35, 36. The positive feed 36 connects to the anode of the source and the negative or ground feed 35 is connected to the cathode. The electrodes feed through walls of the pipe 34 containing exhaust gases 33 to be treated by UV. The UV source 37 emits UV radiation 38 capable of dissociating pollutants and/or contaminants in the gas 33 flowing by the source 37. Reflecting or partially-reflecting walls 39 may be incorporated into the environment in any embodiment in order to return the infrared and visible portion of the spectrum back into the gas environment being treated. Such reflecting walls may be composed of any appropriate metallic substance and may be placed at any reasonable distance from the UV source, typically 20 cm to 50 cm. The distance is limited by the absorption of the UV by the gas environment. The presence of reflecting walls is moot if all of the UV is absorbed by the gas before reaching the walls. In any embodiments, impurities such as but not limited to helium, neon, argon, or xenon can be added to either the fuel or the oxidizer, or both, that can enhance certain portions of the emitted UV spectrum. The choice of impurities can likewise be determined by the required dissociation energies of the contaminants present in the system.

### Modification of the Ultraviolet Radiation Output

The composition and intensity of the UV output of the system can be modified by varying the operation parameters. The optimum output configuration requires tuning all of the parameters described below.

1. Modifying Ultraviolet Output by Varying the Electrical Heating Current

The electrical discharge current is driven through the flame with the application of high voltage to the flame. An initial input of approximately 7 kV to 10 kV is typically required to initiate an electrical discharge through the flame. The magnitude of this voltage is dependent on several parameters such as, but not limited to, flame temperature, flame length, impurity gases, and electrode geometry. The temperature and the resulting UV spectrum can be greatly modified by changing the magnitude of the current input into the flame. Increasing the magnitude of the input current increases the flame temperature (scaling roughly as I²) and vastly increases the level of UV-C radiation. Thus, any operating mode that increases the peak input current can be desirable.

Heating the flame with a pulsed input current source enables the electrical discharge to increase the peak input current. Peak input currents up to several thousand amperes can be achieved by connecting a capacitor having a typical value of 1 μF across the flame, demonstrating improved performance. For example, in the embodiment described in FIG. 5a, a continuous current of 150 mA input into the flame can generate a total UV-C ultraviolet power output of −10 mW. Adding a 1-μF capacitor to the circuit can increase the UV-C ultraviolet power output of the same system up to ˜1 W, while maintaining an average current passing into the flame of 150 mA and the average heating power in the load at ˜600 W to 750 W.

Although not desiring to be bound by any theory of operability, the fundamental efficiency of producing UV-C radiation appears to increase with higher input heating currents, where efficiency is defined as the ratio of the UV-C power out of the system to the electrical power input into the flame. This may be related to the significantly higher temperatures reached with a pulsed current. Increasing the input pulse repetition rate can increase the absolute UV-C power and, hence, the electrical power delivered to the UV source.

2. Modifying Ultraviolet Output by Varying the Capacitor Value

The capacitance value, C, used in the circuit described in FIG. 9 can have a significant impact on the UV-C performance of the system. With no load capacitance, the current through the source is continuous and limited to the peak current of the power supply used. A typical power supply will have a peak voltage of ˜10-15 kV, a current of 0.1 A to 1 A, and deliver a power to the UV source of 1 kW to 10 kW. As the value of the capacitance across the load is increased, the system will begin to operate in a pulsed mode in which the pulse repetition rate decreases, the current pulse width increases, and the peak input current increases. The peak current, I, delivered through the combustion flame increases as C^{1/2}. The limit of operation is a practical one. A capacitor that is too large results in a peak input current that can damage the system electrodes, thereby decreasing system lifetime. Also, for large capacitors and a given power supply, the repetition rate is too low and the system becomes unusable for some waste treatment applications in which the material flow velocity past the source is high.

The system may be operated with different capacitor values. At a capacitance of 0.33 μF, a peak input current of 600 A (at 3.5 kV) has been observed with a repetition rate of 60+Hz and a UVC power output of 0.1 W. With the same system parameters but with a 3.33 μF capacitor, the peak current increased to 5,000 A (at 7.5 kV), the repetition rate decreased to 5 Hz and the average UVC power output increased to 10 W.

3. Modifying Ultraviolet Efficiency and Output by Lowering the Circuit Resistance

The parasitic resistance, R, of the circuit can limit the peak current of the circuit and also can inherently limit the overall electrical efficiency of the circuit. Ideally, the only dissipative element in the circuit should be the resistance of the flame. Lowering the total resistance of the circuit can significantly increase the efficiency of the UV source. Resistance in cables and connections can be avoided by the use of solid copper or brass electrodes. Low-resistance current contacts can be achieved by using carbon-based joint compound. The cables connecting the capacitors to the discharge source can be, for example, #2 gauge copper cable. The copper or brass connecting lugs can be brazed, soldered, or crimped. Circuit resistance (not including the internal capacitor resistance) can be less than 0.1 Ohm, as measured by the discharge source. The RC decay of the current flowing in the circuit can be measured to confirm the total value of resistance in the circuit.

4. Modifying Ultraviolet Output by Controlling Flame Diameter

Reducing the flame diameter can increase the UV output of an electrically heated flame. The simplest way to accomplish this is to reduce the diameter of the nozzle feeding the mixed combustible gas and oxidizer into the flame region, with the effect of increasing the gas density in the flame. By reducing the volume and surface area of the flame, its temperature is increased. At some small diameter this technique will not work as the gas expands rapidly after exiting the nozzle. At a flame diameter of >2 cm, the discharge may not be localized to the entire diameter of the flame and may exhibit a diffuse structure. Reducing the flame diameter to ˜1 cm can greatly improve the UV output and make the discharge through the flame much more uniform. Reducing the flame diameter to ˜0.5 cm can increase the UV output further. Yet smaller flame diameters can result in smaller nozzle electrodes and increased electrode erosion while the flame can expand rapidly away from the nozzle.

Expansion of the combustion gas can be minimized or eliminated by surrounding the flame with an annular column of gas such as nitrogen, oxygen-depleted combustion gas, or air, as seen in See FIG. 6. This annular gas jet is injected via a nozzle at substantially the same physical location as the combustion gas. The flame finds itself confined by the pressure of the annular gas jet. When a high voltage is applied to the flame, the electrical discharge of the system is confined to the volume of the flame gases.

Some reduction in flame diameter is possible by surrounding the flame with a conducting screen and placing on the screen a voltage with negative polarity with respect to the flame. The electrostatic forces have the effect of compressing the combustion flame by ˜10% of its initial diameter. The ability to compress the flame electrostatically is typically limited by the peak voltage that can be applied to the conducting screen without a breakdown.

One desirable combination of the above techniques comprises a nozzle for the combustion gases having a diameter of −0.5 cm confined by a gas jet whose pressure balances the pressure of the combustion flame. Using this configuration, there is little additional benefit in using electrostatic compression when the expense of added complexity is considered.

5. Modifying Ultraviolet Output by Adding Impurity Gases

Depending on the UV requirements and operating cost constraints, the UV spectral intensity of the source can be modified by the addition of impurity gases. These gases can include but are not limited to helium, neon, argon, krypton, and xenon. The impurity gases can be mixed with the combustion gases prior to injection or can be segregated from the flame by injecting these gases in a separate gas jet located internally or externally to the flame. Impurity gases-may be added to the combustion gases in an amount of about 0.1% to 5% by volume. This separate injection of the impurity gases can be made from the cathode (FIGS. 7d & 7e) or the anode (FIG. 8c).

Numerous impurity gases and configurations are suitable for use with the invention, including placing the impurity gases in the combustion flame, around the combustion flame, inside the combustion flame, and inside the combustion flame but injected from the electrode opposite the combustion gas nozzle. One desirable configuration is the injection of argon toward the flame in a separate gas jet flowing from the electrode opposite the nozzle. This configuration gives a higher UV-C ultraviolet output and a lower breakdown voltage.

6. Modifying Ultraviolet Output by Varying the Distance Between the Electrodes

As the distance between the electrodes increases and length of the electrical discharge increases, the magnitude of the UV output of the system also is increased. The electrical efficiency of the high voltage circuit can be increased by increasing the length of the flame because the dissipation of electrical energy in the flame is proportional to the discharge length. The limitation of this approach is the higher voltages required to initiate and drive the electrical discharge. Practical engineering limitations of peak voltage for use in the field push to a maximum input voltage of ˜20 kV. This peak voltage has the effect of limiting the distance between the electrodes and, hence, the length of the discharge to about 5 cm to 7 cm. Additionally, the confined shape of the flame and the high temperature of the flame gases can be lost as flame length increases and mixing of the flame gases with the surrounding gas occurs.

The length of the electrical discharge can be increased by adding argon (or other gases having low ionization thresholds such as helium, neon, krypton, or xenon) to the cooling gas substantially surrounding the anode tube of FIG. 8c and to the cathode combustion gas or the cathode cooling and containment gas. With 20 kV as a practical limit for voltage, argon impurities can extend the potential arc length from 2.5 cm to 6 cm with a doubling of the UV output. Longer discharge lengths are possible by use of any ionization technique that is capable of ionizing a longer path length. For example, a continuous H₂+O₂ flame or the ionization from radioactive materials could be used.

7. Modifying Ultraviolet Output by Using Magnetic Fields

Magnetic fields parallel to the electrical discharge can be used to confine and align the electrical discharge between the electrodes. Even a small magnetic field improves the visual appearance of the electrical discharge, and makes the electrical discharge of the system much more reproducible in the pulsed mode of operation. With a magnetic field up to 500 Gauss using permanent magnets 16 placed around the anode 1 and the cathode 5 as shown in FIG. 5 the UV output of the system is increased slightly (˜10%) over systems without magnets. It is possible to increase the magnitude of the magnetic field further-using electromagnets. Designs that use the discharge current itself in a Helmholtz configuration may provide stabilizing magnetic field at levels >1000 Gauss.

8. Electrode Lifetime

One feature that can limit the lifetime of the UV source is electrode erosion. At large peak currents, electrode material will likely be eroded.

Preferable electrode materials are iridium, tungsten welding rod, brass, and copper, in that order. While expensive, iridium has been observed to experience little or no erosion at the disclosed operating conditions. Tungsten electrodes worked relatively well but have been found to oxidize over a period of time. Copper and brass tubing were observed to work well at low current or if the electrodes are cooled. A steady flow of cooling gas, such as air, can help to greatly reduce the magnitude of electrode erosion even from soft materials such as copper.

One desirable electrode design comprises an outer nozzle (tube) material comprised of copper or brass that is cooled by air. The inner rod tip in the nozzle is preferable for this embodiment if it is fabricated from iridium. For virtually any electrode design, electrode erosion may be observed when the high voltage capacitor is operated at very high current and/or very high repetition rate.

A DC electrical input can be designed so that the peak current delivered to the capacitor connected between the cathode and the anode is limited by transformer design so as to minimize the resistive losses in the power supply. It is also possible to operate the electrical discharge with AC high voltage rather than DC high voltage, and pulse the UV source every 1/120 of a second. The DC voltage, preferably ˜10 kV, can be applied directly to a capacitor, preferably about 3 μF, and the entire circuit can be operated at a repetition rate of ˜10 Hz. The average UV-C power released by a system operated under these conditions is about 10-20 W with an electrical input power of ˜1000 W.

### Treatment of Exhause Gases using the Intense Ultraviolet Radiation Source

One embodiment of the UV system can be applied in treating exhaust gases from a combustion source as shown in FIG. 13. The UV source (or system) 37 can be placed substantially along the axis of an exhaust pipe 34 carrying combustion gases 33 with pollutants and/or contaminants of interest, such as NOₓ. The system 37 can be aligned so that the flame and the resultant electrical discharge of the system 37 runs substantially vertically in the direction of the axis of the exhaust pipe 34, with the flame positioned such that it extends from the nozzle (cathode) to the anode. The flame is substantially in the same direction as the flow of the exhaust gases 33. UV radiation 38 is emitted isotropically from the UV source 37. The exhaust pipe diameter is preferably of a size such that the mean free path of the UV radiation is approximately equivalent to the pipe diameter. UV radiation that is not absorbed by the gases 33 in the pipe 34 may be reflected back into the gases 33 using reflective walls 39. Preferably, the exhaust pipe 34 should be reflective to optimize the use of the UV radiation 38. The particulate content of the exhaust gases 33 may coat the walls and reduce the wall reflectivity with time. The gas 33 flowing in the exhaust pipe 34 can be mixed using vanes so as to move gases flowing along the outside of the pipe to the center of the pipe 34. If the total volume of exhaust gases 33 dictated by the particular application forces the size of the exhaust pipe 34 to be larger than the effective mean free path of the desired UV photons, then multiple UV sources may be required to treat the exhaust gas 33 and a more complex geometry may be required.

The UV source may be exposed directly to the working environment, or it may be contained or otherwise separated from the working environment by an envelope or other barrier, such as a filter, if necessary due to the nature of the working environment or otherwise desired. Similarly, liquid streams may also be treated using the intense UV source.

It is to be understood that the embodiments and variations shown and described herein are merely illustrative of the principles of this invention and that various modifications may be implemented by those skilled in the art without departing from the scope and spirit of the invention.

We claim:

1. An intense ultraviolet radiation source comprising:
a combustion flame exiting an orifice, the combustion flame having a longitudinal axis;
at least two electrodes, wherein at least one electrode is a cathode and at least one electrode is an anode, wherein the cathode and the anode are located substantially at opposite ends of the longitudinal axis of the combustion flame; and
an electrical input discharged into the combustion flame,
wherein the electrical input is capable of causing a current to pass between the cathode and the anode

2. The intense ultraviolet source of claim 1, wherein the orifice comprises a nozzle.

3. The intense ultraviolet source of claim 1, wherein at least one electrode comprises a hollow tube, needle, or mesh.

4. The intense ultraviolet source of claim 1, wherein fuel for combustion of the flame comprises diesel fuel, hydrogen, acetylene, propane, butane, or methane, or mixtures thereof.

5. The intense ultraviolet source of claim 4, wherein the fuel feed rate is approximately 1 to 3 cm³/min.

6. The intense ultraviolet source of claim 4, wherein the fuel further comprises an oxidizer.

7. The intense ultraviolet source of claim 6, wherein the oxidizer comprises oxygen, fluorine, chlorine, or air, or mixtures thereof.

8. The intense ultraviolet source of claim 1, wherein the cathode is in contact with the orifice.

9. The intense ultraviolet source of claim 1, wherein the orifice comprises the cathode.

10. The intense ultraviolet source of claim 1, wherein the anode is approximately about 1 cm to about 10 cm from the cathode.

11. The intense ultraviolet source of claim 10, wherein the anode is approximately about 5 cm from the cathode.

12. The intense ultraviolet source of claim 1, wherein the electrical input comprises a voltage of about 1 kV to about 20 kV applied substantially to the cathode.

13. The intense ultraviolet source of claim 12, wherein the electrical input comprises a voltage of about 10 kV applied substantially to the cathode.

14. The intense ultraviolet source of claim 1, wherein the electrical input comprises a pulsed current discharge.

15. The intense ultraviolet source of claim 1, wherein a capacitor is connectively placed between the anode and the cathode.

16. The intense ultraviolet source of claim 1, further comprising an external electromagnetic radiation source.

17. The intense ultraviolet source of claim 16, wherein the external electromagnetic radiation source comprises a laser, a microwave generator, or a radio-frequency induction heater.

18. The intense ultraviolet source of claim 1, further comprising a focusing grid.

19. The intense ultraviolet source of claim 18, further comprising a magnetic field.

20. The intense ultraviolet source of claim 1, further comprising an annular gas jet substantially surrounding the orifice.

21. The intense ultraviolet source of claim 1, further comprising impurities in the combustion flame, wherein the impurities comprise helium, neon, argon, and xenon or combinations thereof.

22. The intense ultraviolet source of claim 21 wherein the impurities are added to at least one of the fuel or the oxidizer.

23. The intense ultraviolet source of claim 1, further comprising reflecting walls.

24. The intense ultraviolet source of claim 1, wherein at least one electrode is cooled by a cooling gas flow.

25. The intense ultraviolet source of claim 25, wherein the cooling gas flow comprises impurities, which include helium, neon, argon, and xenon or combinations thereof.

26. The intense UV source of claim 1, wherein the combustion flame emits at least about 500 W per meter length of the combustion flame.

27. The intense UV source of claim 26, wherein the combustion flame emits at least about 1000 W per meter length of the combustion flame.

28. The intense UV source of claim 26, wherein the combustion flame emits at least about 2000 W per meter length of the combustion flame.

29. The intense UV source of claim 26, wherein there is no barrier between the UV source and a working environment in which the UV source is placed.

30. A method for creating an intense ultraviolet source comprising:
creating a combustion flame; and
discharging an electrical input into the combustion flame;
wherein the combustion flame is substantially aligned axially between two electrodes such that the electrical input causes an electrical discharge between the-two electrodes.

31. The method of claim 30, wherein the electrical input comprises a pulse discharged into the combustion flame.

32. The method of claim 31, wherein the electrical input is discharged substantially into one of the electrodes, wherein further the electrodes are connected by a capacitor.

33. The method of claim 32, further comprising adding impurities to the combustion flame, wherein the impurities comprise helium, neon, argon, and xenon or combinations thereof.

34. The method of claim 33, further comprising intensifying the combustion flame by use of a focusing grid, a magnetic field, or an annular gas jet flow.

35. The method of claim 34, further comprising an auxiliary electrical input comprising an external electromagnetic radiation source.

36. The method of claim 35, wherein the external electromagnetic radiation source comprises a laser, a microwave generator, or a radio-frequency induction heater.

37. The method of claim 30, wherein the combustion flame emits at least about 1000 W per meter length of the combustion flame.

38. A method of causing the molecular dissociation of contaminants, pollutants or a combination of contaminants and pollutants comprising:
placing an intense ultraviolet source substantially in an axial direction to the flow of the contaminants, pollutants or a combination of contaminants and pollutants; and
exposing contaminants and/or pollutants to the radiation of a combustion flame, wherein the intense ultraviolet source is formed by the combustion flame, which is aligned substantially axially between two electrodes; and
discharging an electrical input such that the electrical input causes an electrical discharge between the two electrodes substantially through at least a portion of the combustion flame,

39. The method of claim 38 wherein the contaminants, pollutants or the combination of contaminants and pollutants comprise at least one of nitric oxides, nitrogen dioxides, nitrous oxides, sulfur oxides, carbon monoxide, carbon dioxide.

40. The method of claim 38, wherein the electrodes are connectively related by a capacitor.

41. The method of claim 38, wherein the combustion flame is capable of emitting at least about 500 W per meter length of the combustion flame.

42. The method of claim 41, wherein the combustion flame is capable of emitting at least about 1000 W per meter length of the combustion flame.

43. The method of claim 42, wherein the combustion flame is capable of emitting at least about 2000 W per meter length of the combustion flame.

44. The method of claim 38, wherein there is no barrier between the UV source and the contaminants, pollutants or the combination of contaminants and pollutants.

45. A system to treat contaminants, pollutants or a combination of contaminants and pollutants by molecular dissociation, comprising:
an intense ultraviolet source; and
a conduit capable of carrying a stream containing the contaminants, pollutants or combination of contaminants and pollutants; wherein the intense ultraviolet source comprises:
a combustion flame having a longitudinal axis; at least two electrodes, wherein at least one electrode is a cathode and wherein at least one electrode is an anode, wherein the cathode and the anode are located substantially at opposite ends of the longitudinal axis of the combustion flame; and
an electrical input discharged into the combustion flame,
wherein the electrical input is capable of causing a current to pass substantially between the cathode and the anode, wherein further the contaminants, pollutants or the combination of contaminants and pollutants in the pipe are exposed to the radiation output of the combustion flame.

46. The system of claim 45 wherein there is no barrier between the UV source and the contaminants, pollutants or the combination of contaminants and pollutants.

47. The system of claim 45 wherein the combustion flame is capable of emitting at least about 500 W per meter length of the combustion flame.

48. The system of claim 47 wherein the combustion flame is capable of emitting at least about 1000 W per meter length of the combustion flame.

49. The system of claim 48 wherein the combustion flame is capable of emitting at least about 2000 W per meter length of the combustion flame.

50. A system to treat contaminants, pollutants or a combination of contaminants and pollutants by chemical reaction, comprising:
an intense ultraviolet source; and
a conduit capable of carrying a stream containing the contaminants, pollutants or combination of contaminants and pollutants;
wherein the intense ultraviolet source comprises:
a combustion flame having a longitudinal axis;
at least two electrodes, wherein at least one electrode is a cathode and wherein at least one electrode is an anode, wherein the cathode and the anode are located substantially at opposite ends of the longitudinal axis of the combustion flame; and
an electrical input discharged into the combustion flame,
wherein the electrical input is capable of causing a current to pass substantially between the cathode and the anode, wherein further the contaminants, pollutants or the combination of contaminants and pollutants in the pipe are exposed to the radiation output of the combustion flame.

51. The system of claim 50 wherein there is no barrier between the UV source and the contaminants, pollutants or the combination of contaminants and pollutants.

52. The system of claim 50 wherein the combustion flame is capable of emitting at least about 500 W per meter length of the combustion flame.

53. The system of claim 52 wherein the combustion flame is capable of emitting at least about 1000 W per meter length of the combustion flame.

54. The system of claim 53 wherein the combustion flame is capable of emitting at least about 2000 W per meter length of the combustion flame.
